(19) United States
(12) Patent Application Publication
(10) Pub. No.: US 2012/0088964 A1
(43) Pub. Date: Apr. 12, 2012

(54) Methods and devices for pericardial access

(75) Inventors: Kabir GAMBHIR, San Diego, CA (US); Thomas A. RITTER, San Marcos, CA (US)

(73) Assignee: EPICARDIAL TECHNOLOGIES, INC., San Clemente, CA (US)

(21) Appl. No.: 12/902,131

(22) Filed: Oct. 11, 2010

(51) Int. Cl. A61B 1/00

(52) U.S. Cl. 600/104

(57) ABSTRACT

Methods and devices for pericardial access are described. One embodiment of a device includes a distal portion with a visualization element, along with a retractable access element. The distal portion is inserted into and navigates through the body to locate the pericardium with the aid of the visualization element. The access element is then extended and used to create an access site in the pericardium, again with the aid of the visualization element. The methods and devices may also be used to access other internal cavities, other soft tissues and organs, and the mediastinal space.

### BACKGROUND

The pericardium is a tough, fibrous sac which surrounds and protects the heart. The pericardial space is formed between the two layers of the pericardium, the parietal pericardium and the serous pericardium. The serous pericardium has two layers, the first a fibrous layer and the second the epicardium which is closest to the heart. Pericardial fluid within the pericardial space serves to lubricate the motion of the heart.

The pericardial space may be accessed to treat the heart for any one of a number of conditions. For example, the pericardial space may be accessed to perform epicardial ablations for the treatment of arrhythmias such as atrial fibrillation. The pericardial space may also be accessed to deliver drugs and stem cells for the treatment of heart attacks.

The pericardial space may be accessed using minimally invasive techniques. One common technique involves guiding a needle to the pericardium, and then advancing the needle through the pericardium, all under fluoroscopy. However, because of anatomical variations and previous procedures, it may take up to an hour to navigate less than 10 cm through the body to locate a suitable area on the pericardium to create an access site. Navigating through the body with a sharp needle creates the risk of causing damage to structures such as the liver. During pericardial access, the risk posed by a sharp needle may cause damage to the underlying structures such as the coronary arteries and myocardium.

The mediastinal space is the region between the two pleural sacs, with the sternum in front and the vertebral column behind. The mediastinal space can be an especially difficult area to access, especially in the area posterior of the heart, superior to the diaphragm, and inferior to the clavicle.

What is needed are methods and devices which will reduce the amount of time needed to locate the pericardium, and reduce the risk of unintended puncture or damage to other structures during the location process.

What is also needed are methods and devices which will facilitate the creation of an access site through the pericardium, while reducing the risk of damage or irritation to underlying structures.

What is also needed are methods and devices which will facilitate access to the mediastinal space.

### SUMMARY

In one embodiment, a device for accessing a pericardial space includes a housing having a handle, a central portion coupled to the handle, a distal portion coupled to the central portion, and a longitudinal access lumen. The central portion and the distal portion are configured to be inserted through a percutaneous puncture and navigate within a body. The device also includes an access element at least partially positioned within the access lumen. The access element is capable of being extended and retracted from a distal end of the distal portion of the housing. The access element is capable of penetrating a pericardial membrane surrounding the pericardial space to create an access site for accessing the pericardial space. The device also includes a visualization element coupled to the distal portion of the housing. The visualization element is capable of aiding navigation within the body and visualizing the access element.

### BRIEF DESCRIPTION OF THE DRAWINGS

FIGS. 1A-1D shows one embodiment of an access device 100.

FIGS. 2A-2F show one method of using access device 100.

FIGS. 3A-3D show another embodiment of an access device 200.

FIGS. 4A-4G show one method of using access device 200.

FIGS. 5A-5D show yet another embodiment of an access device 300.

FIGS. 6A-6G and 7A-7D show one method of using access device 300.

FIGS. 8A-8C show other embodiments of a distal portion of access device 300.

FIGS. 9A-9D show still another embodiment of an access device 400.

### DETAILED DESCRIPTION

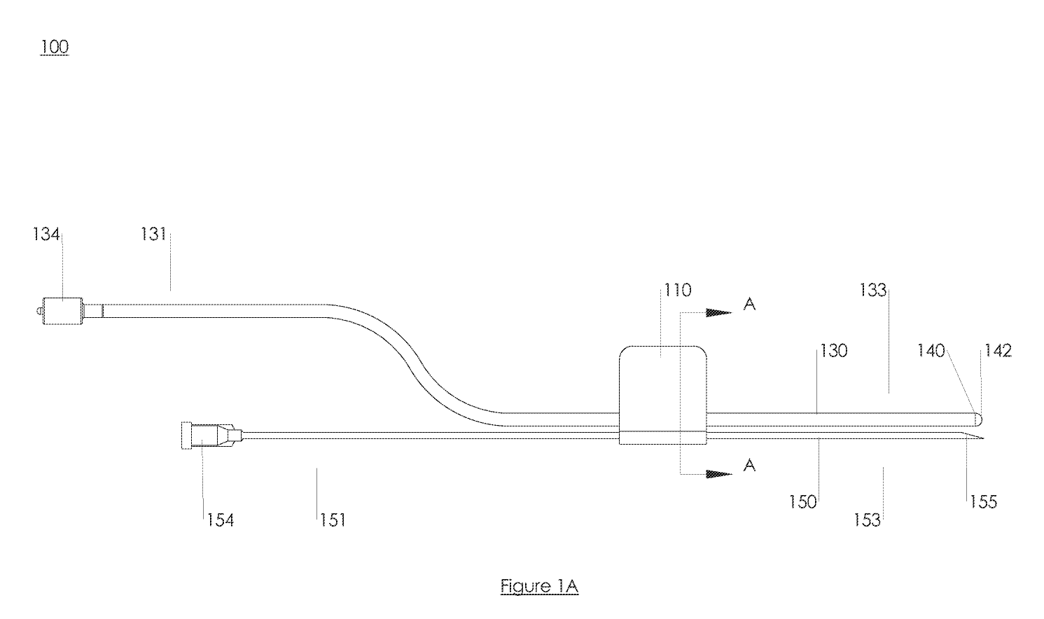

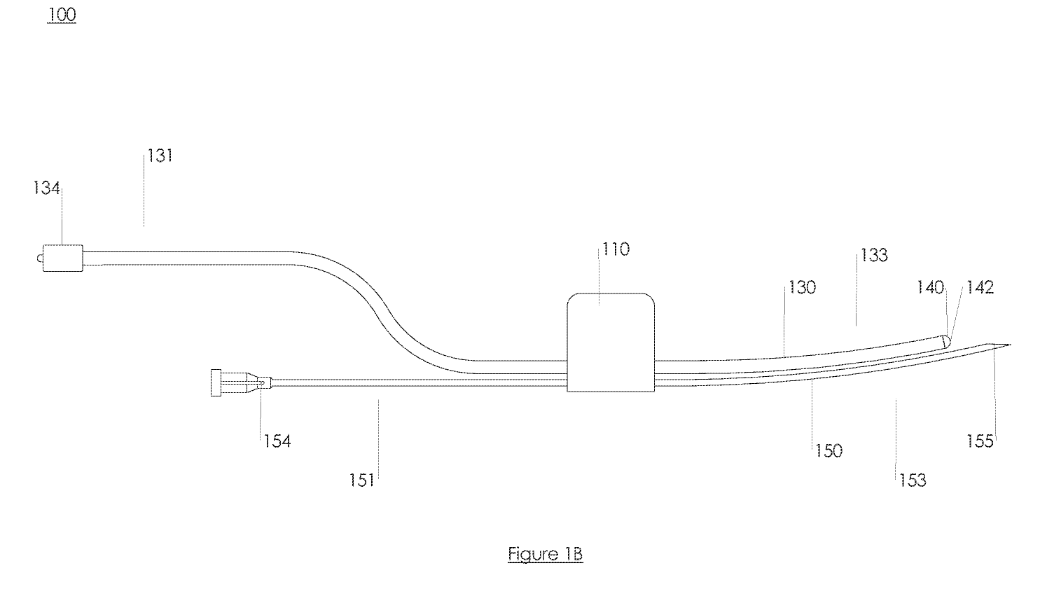

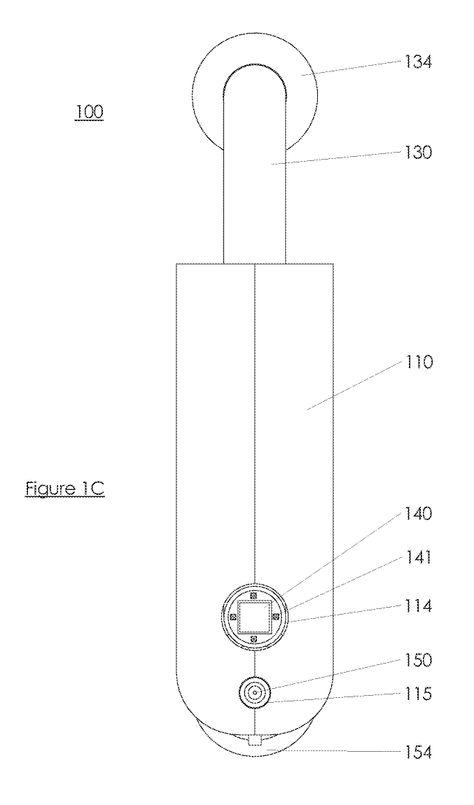

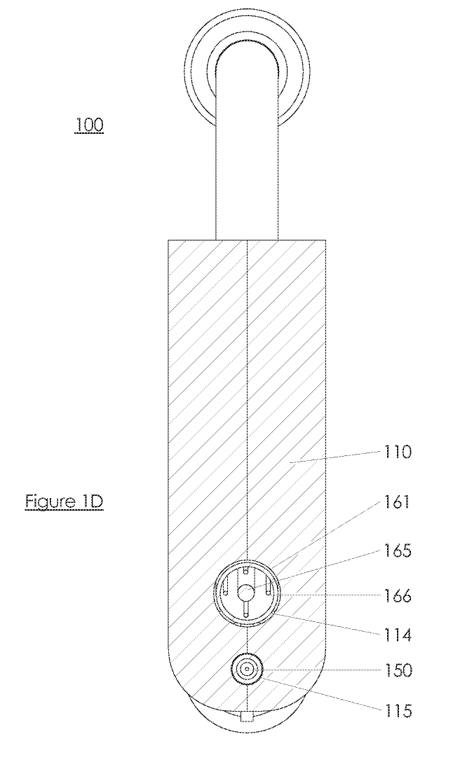

FIGS. 1A-1B show side views of one embodiment of an access device 100. FIG. 1C shows an end view of access device 100. FIG. 1D shows a cross-sectional end view of access device 100.

Access device 100 includes a handle 110, a visualization catheter 130 with a visualization element 140, and an access element 150.

Handle 110 includes a catheter lumen 114 and an access lumen 115. Handle 110 may be constructed as two halves or as a clamshell.

Visualization catheter 130 is at least partially positioned within catheter lumen 114, and can slide and rotate within catheter lumen 114. Visualization catheter 130 includes a proximal portion 131 and a distal portion 133. Visualization catheter 130 may be a hollow tube made of a ductile material such as stainless steel or any other suitable material. Visualization catheter 130 includes a lumen 161. Proximal portion 131 may be configured to facilitate rotation of visualization catheter 130 within catheter lumen 114. Proximal portion 131 may be configured with an S-shaped bend to facilitate manipulation of visualization catheter 130. Proximal portion 131 may include a coupling 134 for attachment of a power source and a video monitor. Distal portion 133 includes a visualization element 140 and one or more lights 141.

Visualization element 140 and lights 141 may be coupled to the tip or end of distal portion 133. Alternatively, visualization element 140 and lights 141 may be coupled to the side or any other suitable location of distal portion 133. Visualization element 140 and lights 141 are coupled to visualization wires 165 and light wires 166 which pass through lumen 161 to coupling 134. Visualization element 140 and lights 141 are covered by a lens 142. Lens 142 may have a hydrophobic coating or other coating to reduce adhesion of natural and synthetic materials that would obscure the image. As shown in FIG. 1B, distal portion 133 may have a curved configuration, and may be bent or otherwise configured by the user and hold its shape.

Access element 150 is at least partially positioned within access lumen 115, and can slide and rotate within access lumen 115. Access element 150 may be used for injection of a liquid, passing of a guidewire 105, application of a vacuum, or any other suitable purpose. Access element 150 includes a proximal portion 151 and a distal portion 153. Proximal portion 151 may include a coupling 154. Distal portion 253 has a tip 255 that may be a blunt tip trocar, a blunt tip obturator, a sharp edge trocar, a sharp edge needle (e.g., Tuohy, epidural, biopsy), a guidewire tip, or any other suitable instrument. Access element 150 may be configured to work with an RF, microwave, cryoablation, high intensity focused ultrasound (HIFU), laser, or any other suitable energy source. Distal portion 153 may have depth markings. Distal portion 153 may be connected to an ohmmeter to measure impedance as the needle penetrates the pericardial membrane into the pericardial space. The impedance measurement may be used to provide an indication as to whether the pericardial membrane has been penetrated. As shown in FIG. 1B, distal portion 153 may have a curved configuration, and may be bent or otherwise configured by the user and hold its shape.

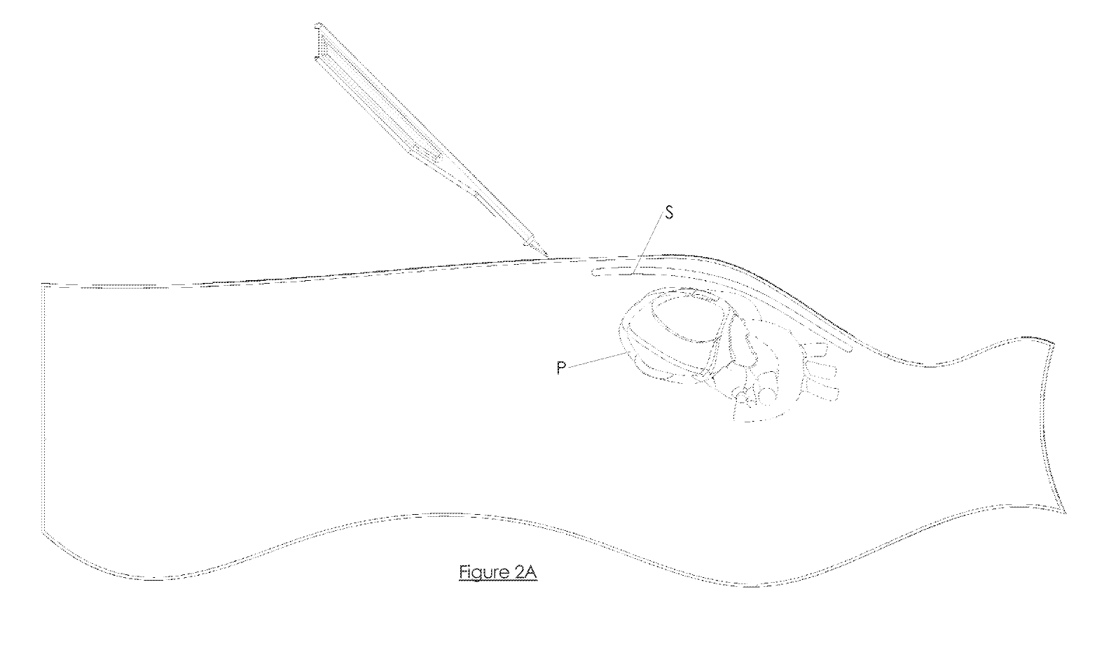

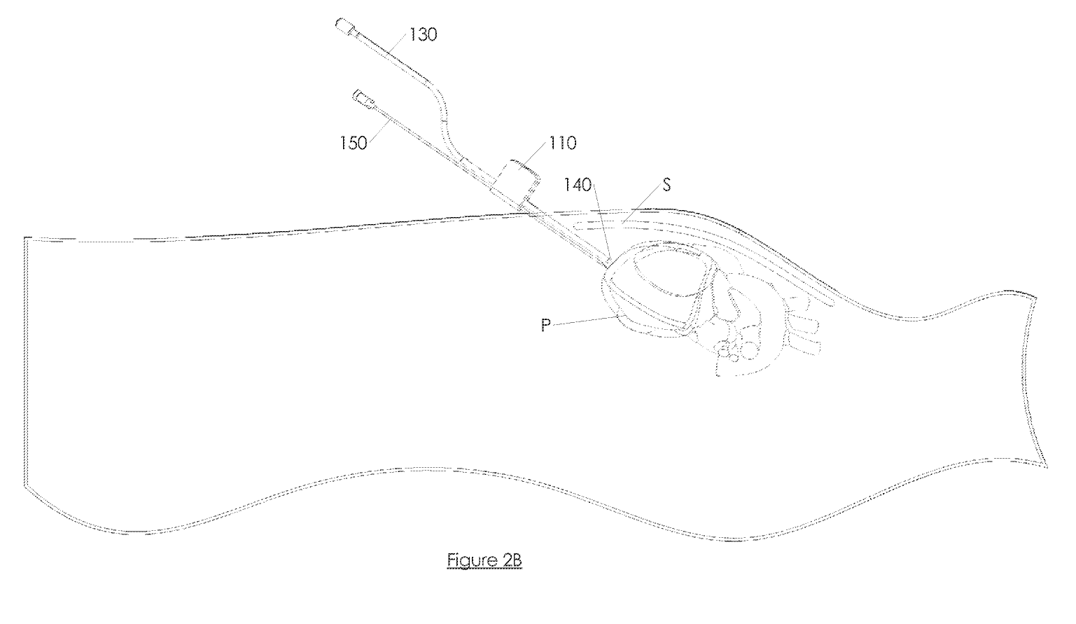

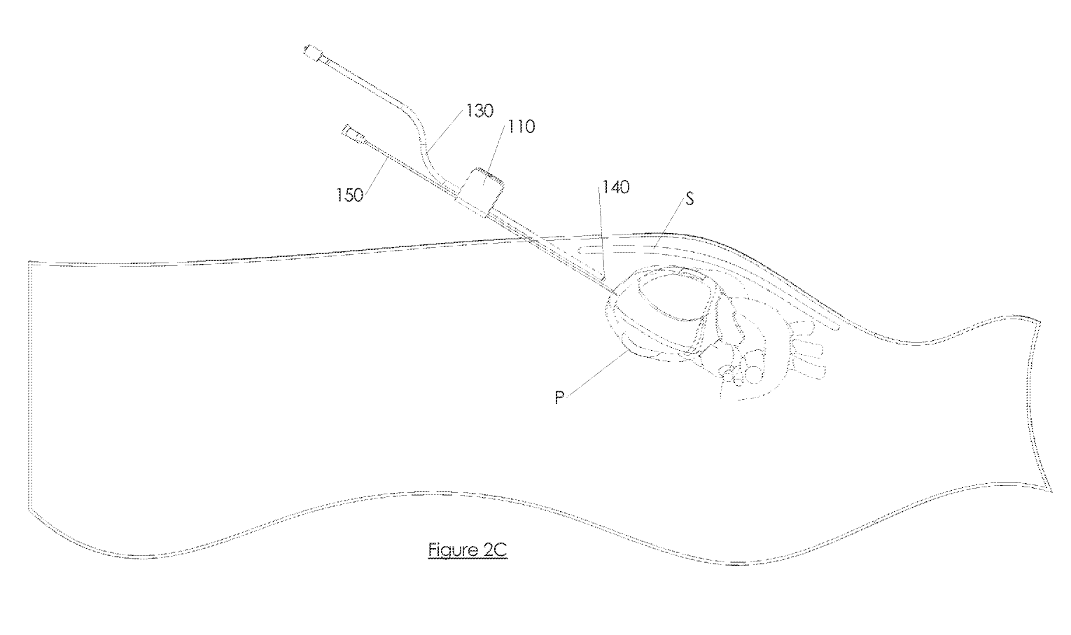

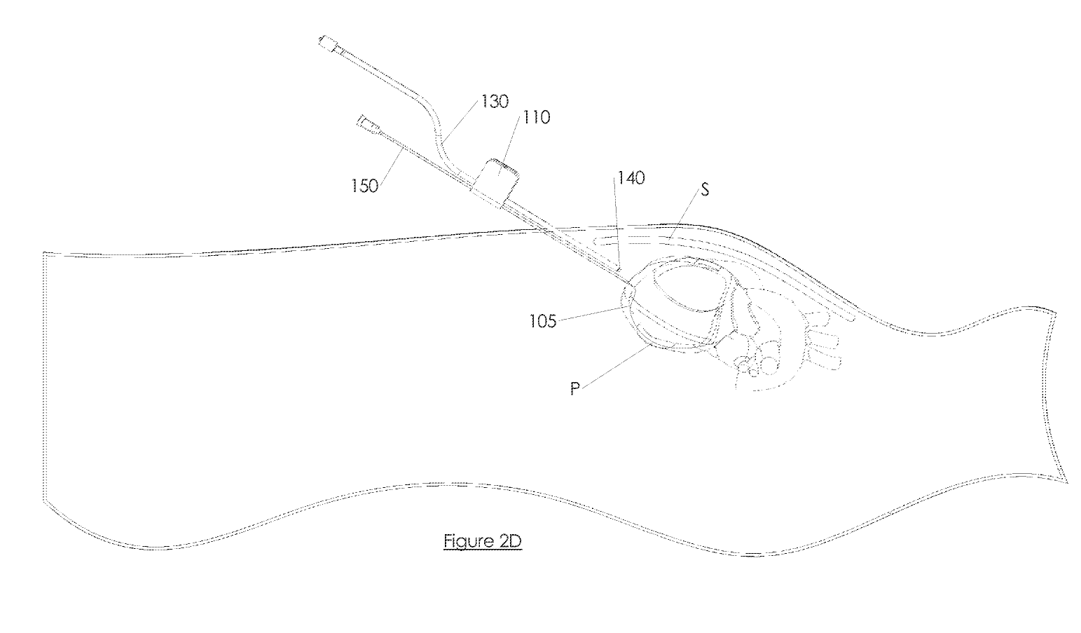

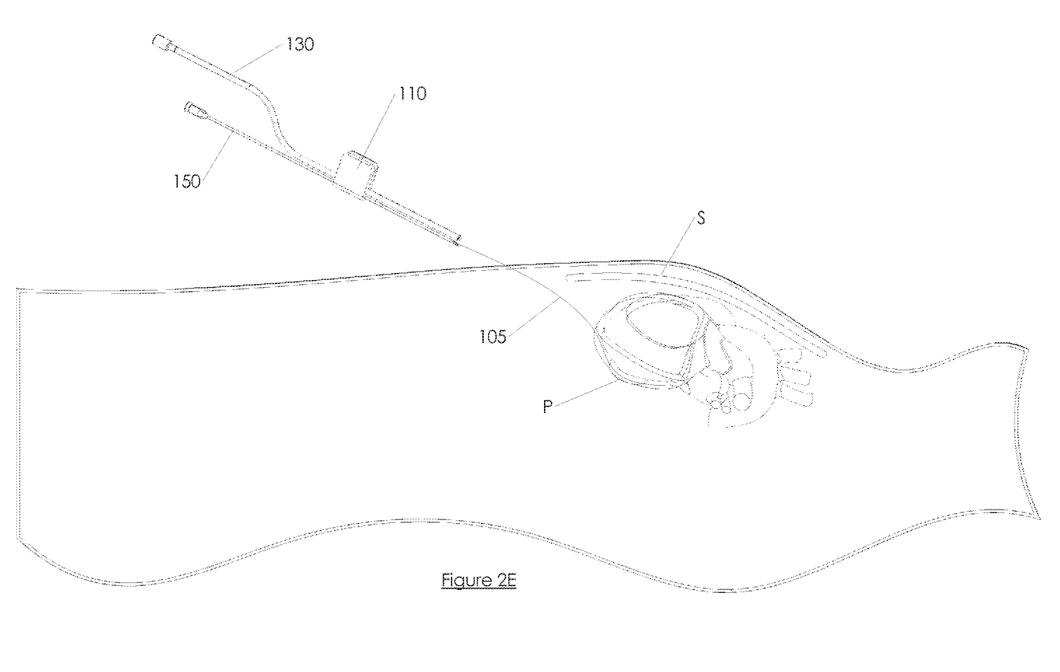

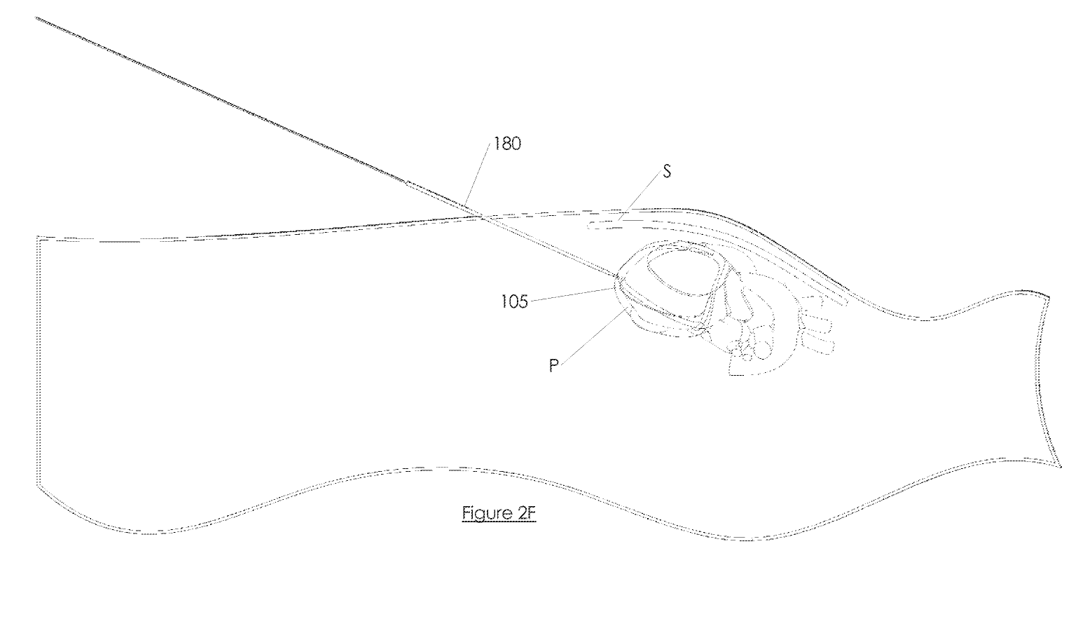

FIGS. 2A-2F show one method of using access device 100.

FIG. 2A shows a percutaneous puncture being made for a subxiphoid approach. Alternatively, an intercostal, apical, subclavian, suprasternal, or any other suitable approach may be used.

FIG. 2B shows visualization catheter 130 and access element 150 inserted through the puncture and positioned at or near the surface of the pericardium P. Visualization element 140 is used to guide visualization catheter 130 and access element 150 along the posterior aspect of the sternum S to the surface of the pericardium P. Visualization catheter 130 may be rotated and moved in and out.

FIG. 2C shows access element 150 advanced through pericardium P to create an access site. For an access element 150 having a sharp tip 155, visualization element 140 may be used to visualize access element 150 as it is advanced through pericardium P. For an access element 150 used with RF energy, visualization element 140 may be used to visualize access element 150 as RF energy is passed through access element 150 to penetrate pericardium P. Access element 150 may be rotated so that a desired surface is visible to visualization element 140. Saline, contrast, medications, and/or other fluids may be introduced through access element 150 into the pericardial space.

FIG. 2D shows guidewire 105 passed through access element 150 and positioned in the pericardial space.

FIG. 2E shows visualization catheter 130 and access element 150 withdrawn, leaving guidewire 105 in place.

FIG. 2F shows a sheath 180 advanced over guidewire 105 through the puncture and the access site and into the pericardial space. Other devices or guidewires may be advanced through sheath 180 to access the pericardial space. Saline, contrast, medications, and/or other fluids may be introduced through sheath 180 into the pericardial space.

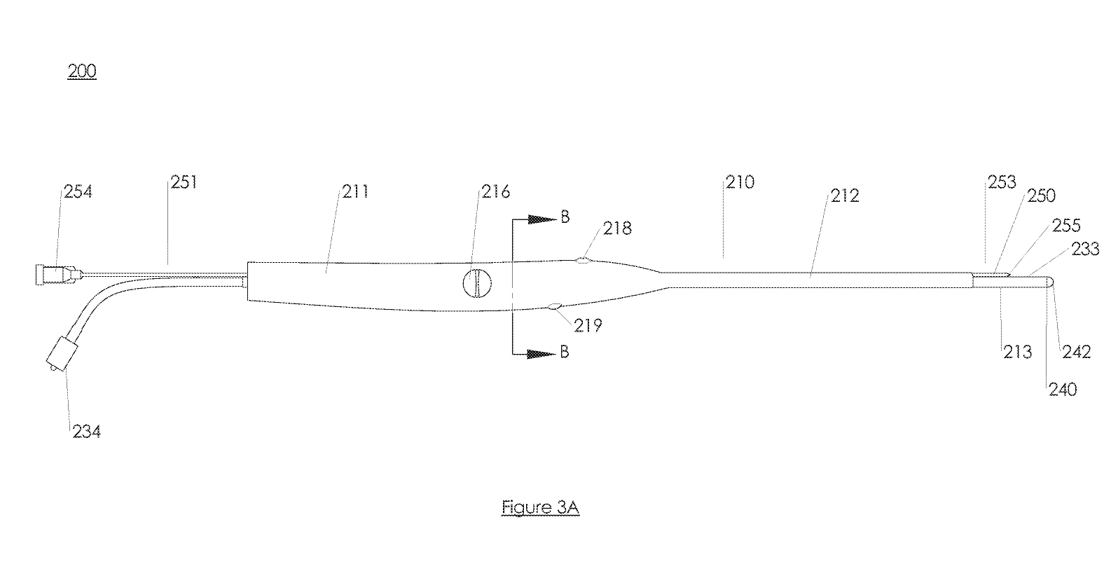

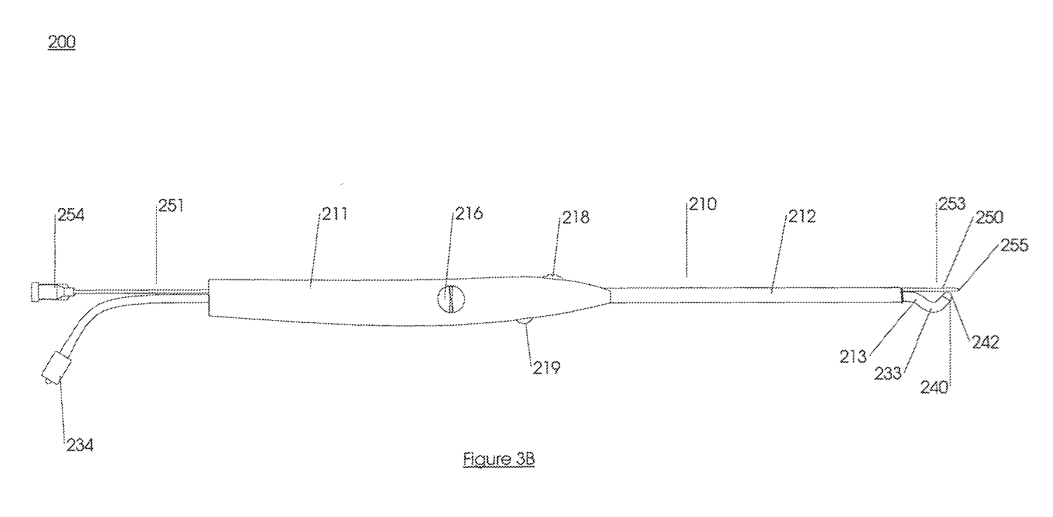

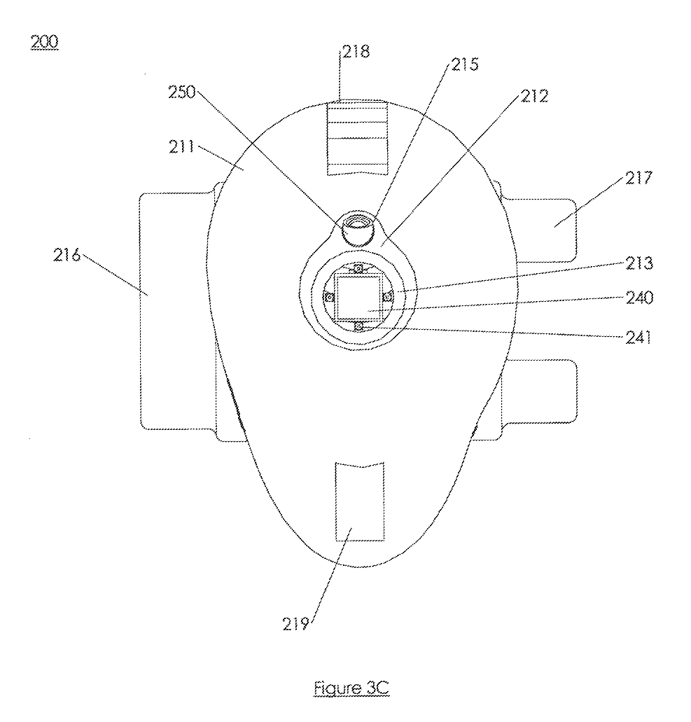

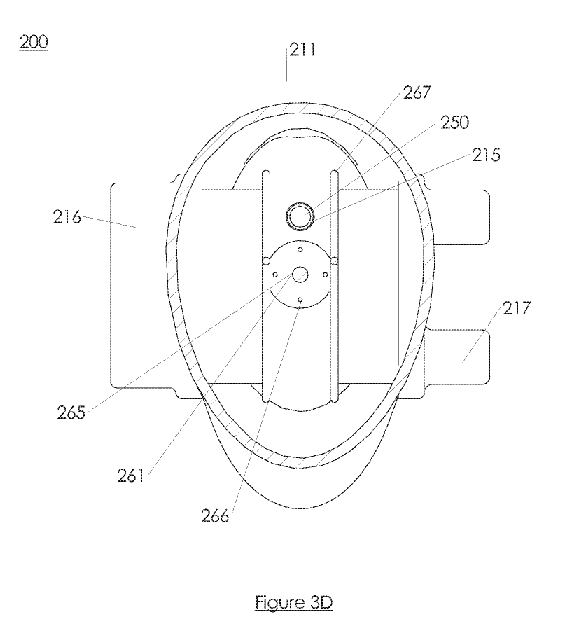

FIGS. 3A-3B show side views of another embodiment of an access device 200. FIG. 3C shows an end view of access device 200. FIG. 3D shows a cross-sectional end view of access device 200.

Access device 200 includes a housing 210, a visualization element 240, and an access element 250.

Housing 210 includes a handle 211, a central portion 212, and a deflectable portion 213. Housing 210 also includes an access lumen 215 and a visualization lumen 261. Handle 211 includes a steering control 216, a tension lock 217, a visualization control 218, and a light control 219. Handle 211 may also include a coupling 234 for attachment of a power source and a video monitor. Central portion 212 is coupled to handle 211, and is configured to be inserted into a puncture and navigate inside the body. Central portion 212 may be soft and flexible, or more rigid depending on the application and user preferences. Central portion 212 and/or deflectable portion 213 may have a cross-section that has a keyhole shape or any other suitable shape.

Deflectable portion 213 is coupled to central portion 212 and is also configured to be inserted into a puncture and navigate inside the body. Deflectable portion 213 may be deflected in one or more axes, as shown for example in FIG. 3B. Deflectable portion 213 may be controlled with pullwires 267 coupled to steering control 216. Deflectable portion 213 may be locked in a desired configuration using tension lock 217. Deflectable portion 213 includes a visualization element 240 and one or more lights 241.

Visualization element 240 and lights 241 may be coupled to a distal end 233 of deflectable portion 213. Alternatively, visualization element 240 and lights 241 may be coupled to the side or any other suitable location of deflectable portion 213. Visualization element 240 and lights 241 are coupled to visualization wires 265 and light wires 266 which pass through visualization lumen 261 to coupling 234. Visualization element 240 and lights 241 are covered by a lens 242. Lens 142 may have a hydrophobic coating or other coating to reduce adhesion of natural and synthetic materials that would obscure the image. Visualization element 240 may be turned on or off, or capture turned on or off using visualization control 218. Lights 241 may be turned on or off, or their intensity adjusted using light control 219.

Access element 250 is at least partially positioned within access lumen 215, and can slide and rotate within access lumen 215. Access element 250 may be used for injection of a liquid, passing of a guidewire 205, application of a vacuum, or any other suitable purpose. Access element 250 includes a proximal portion 251 and a distal portion 253. Proximal portion 251 may include a coupling 254. Distal portion 253 has a tip 255 that may be a blunt tip trocar, a blunt tip obturator, a sharp edge trocar, a sharp edge needle (e.g., Tuohy, epidural, biopsy), a guidewire tip, or any other suitable instrument. Access element 250 may be configured to work with an RF, microwave, cryoablation, high intensity focused ultrasound (HIFU), laser, or any other suitable energy source. Distal portion 253 may have depth markings. Distal portion 253 may be connected to an ohmmeter to measure impedance as the needle penetrates the pericardial membrane into the pericardial space. The impedance measurement may be used to provide an indication as to whether the pericardial membrane has been penetrated. Access element 250 may be moved and rotated by manipulating proximal portion 251.

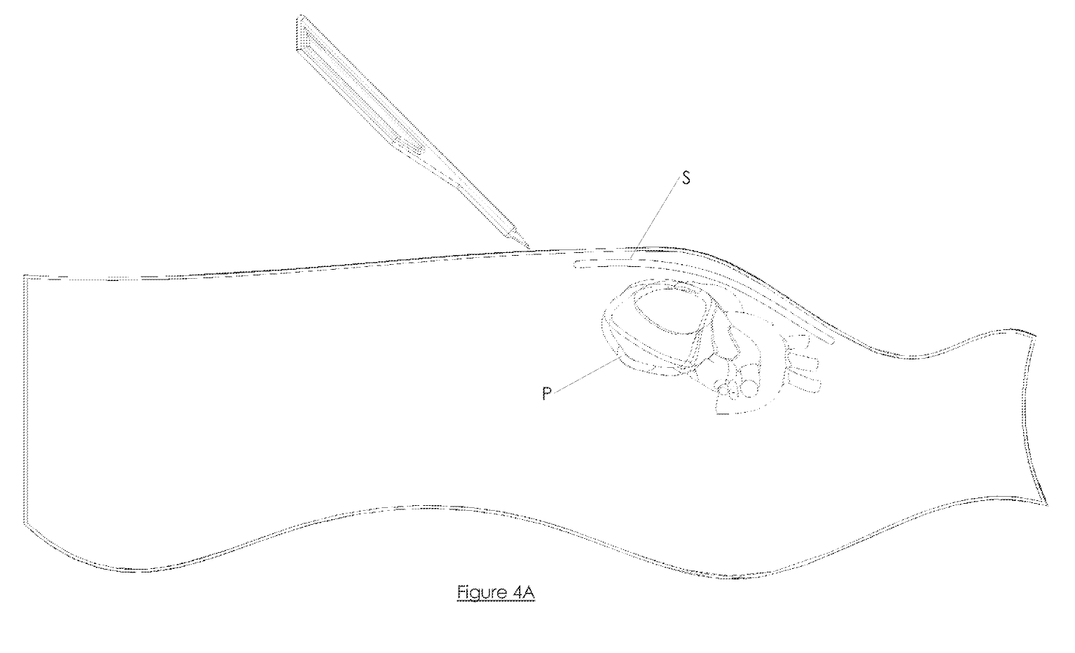

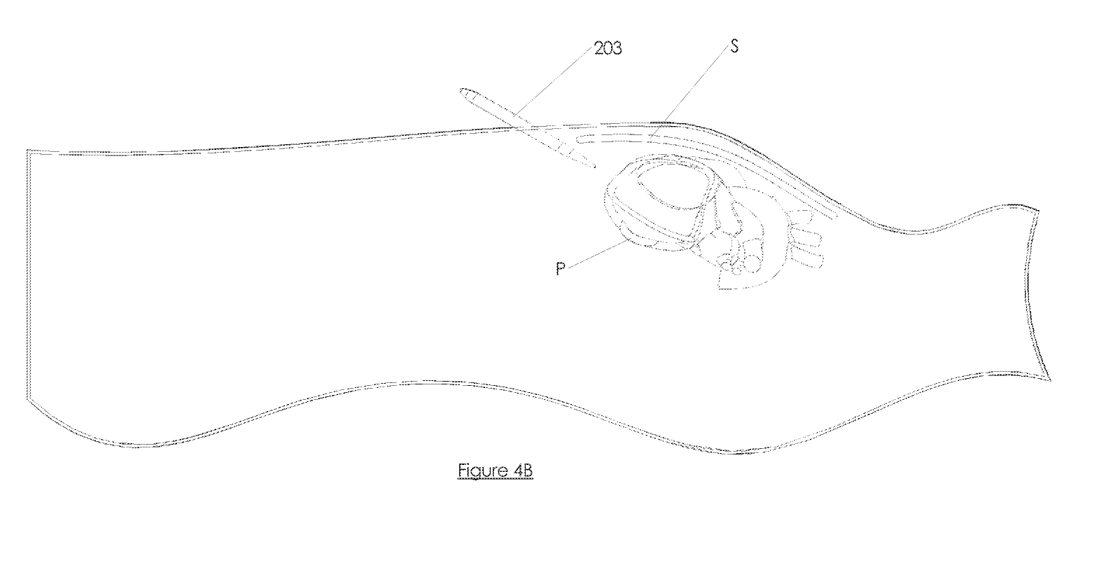

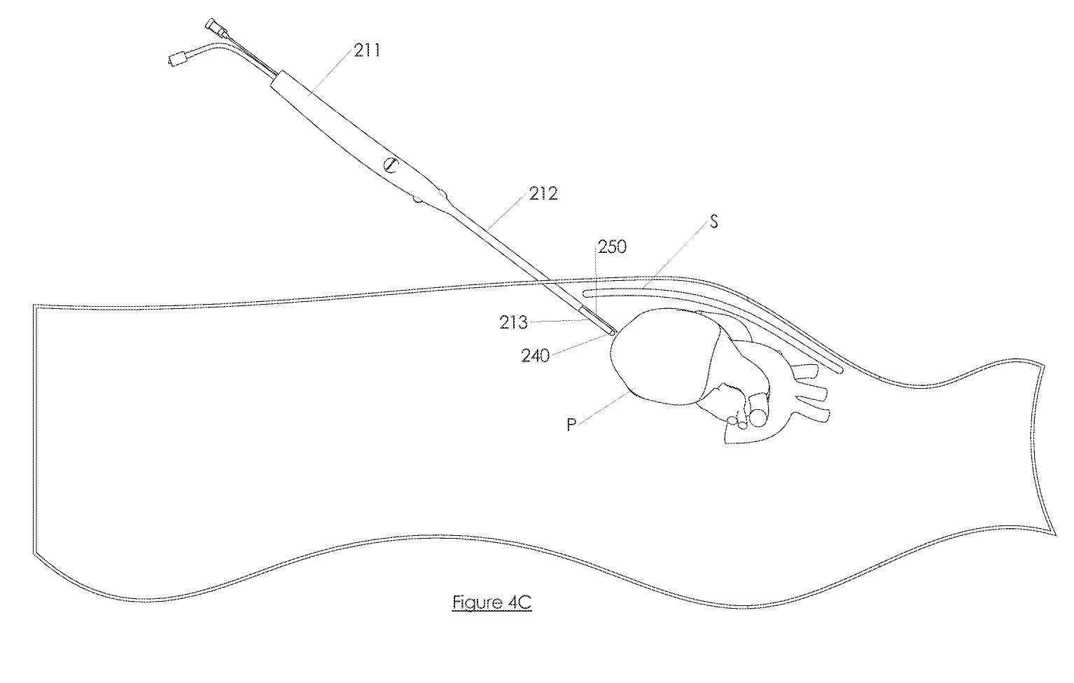

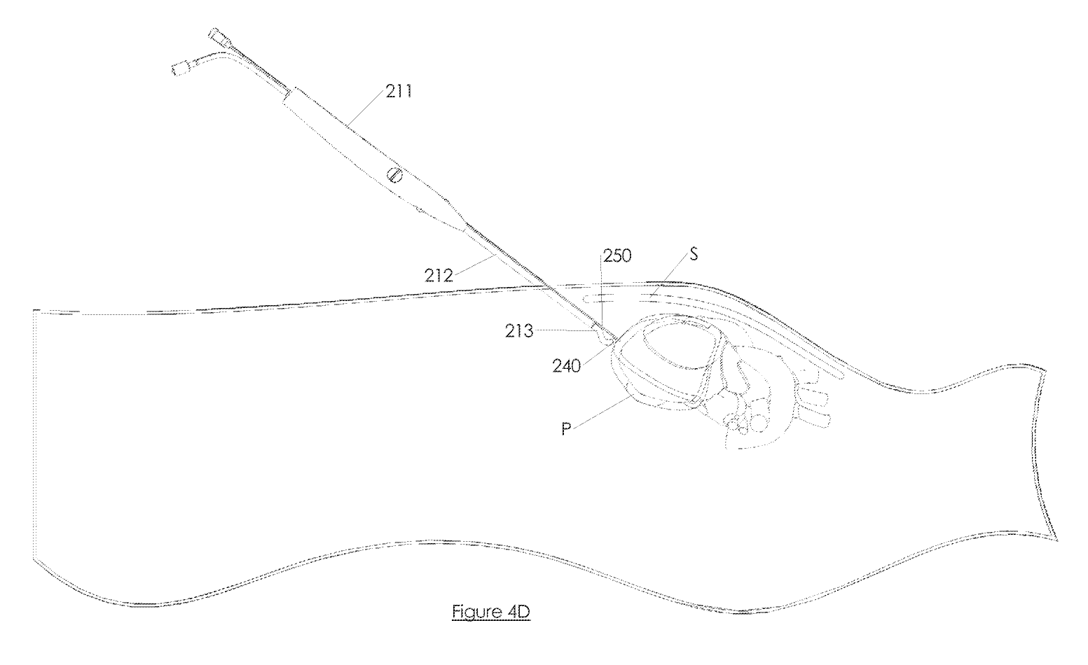

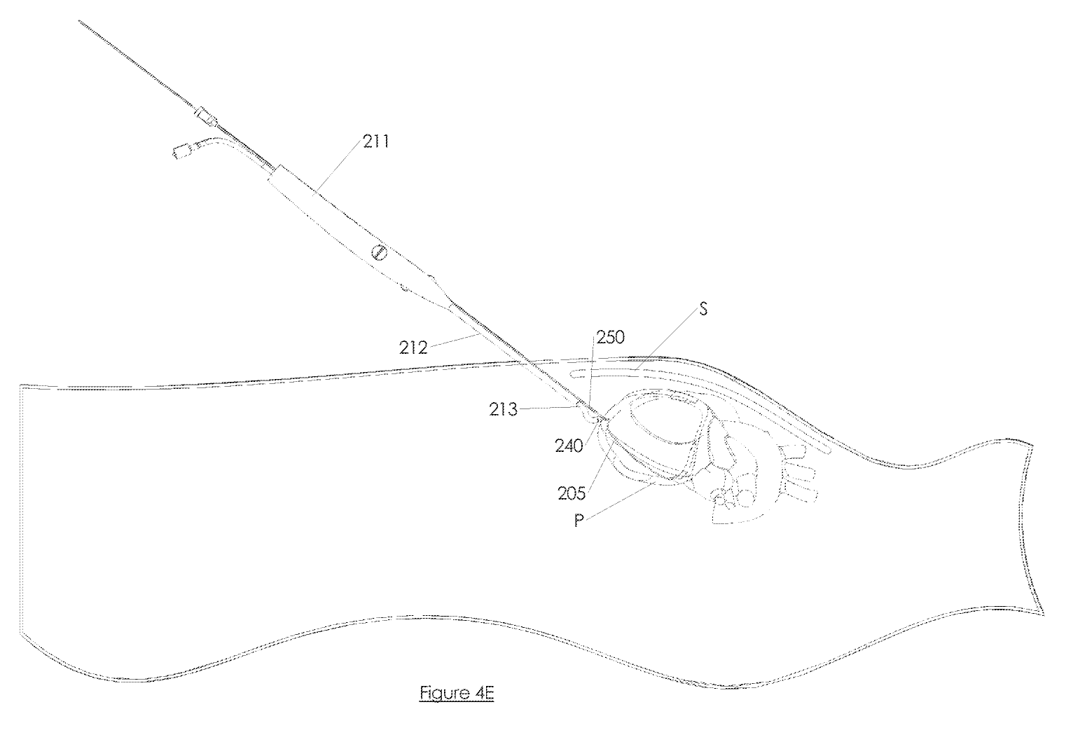

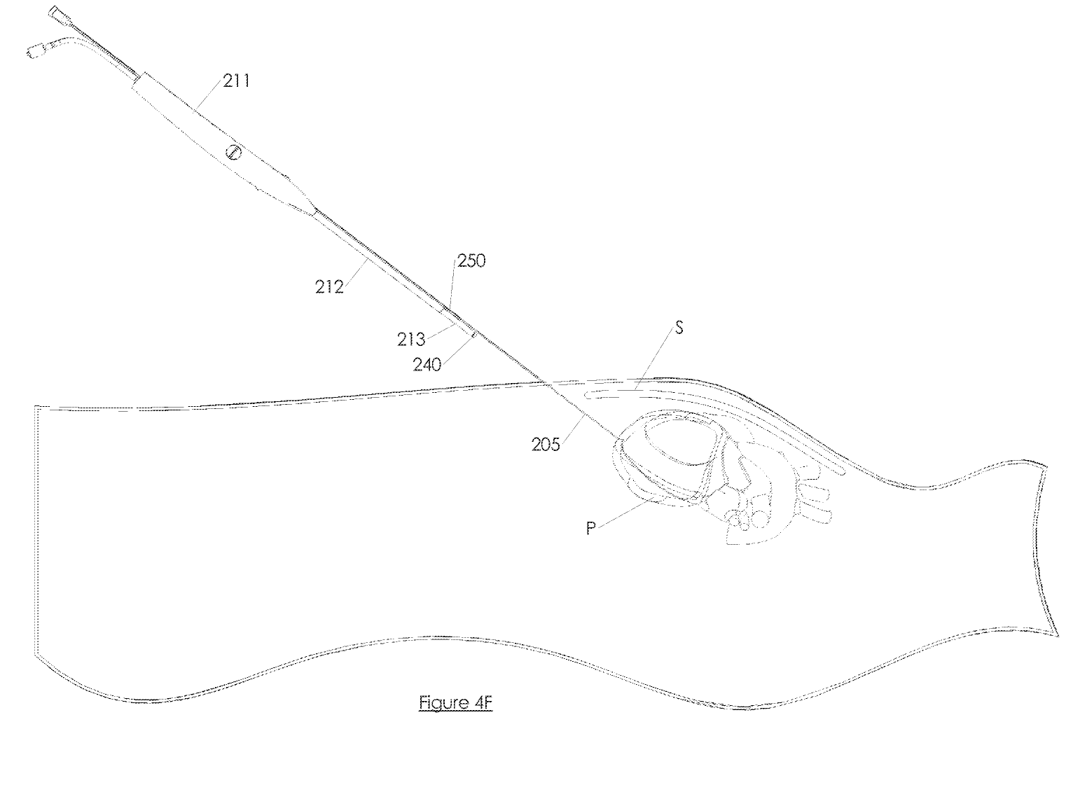

FIGS. 4A-4G show one method of using access device 200.

FIG. 4A shows a percutaneous puncture being made for a subxiphoid approach. Alternatively, an intercostal, apical, subclavian, suprasternal, or any other suitable approach may be used.

FIG. 4B shows a dilator 203 inserted through the puncture. Dilator 203 is used to dilate the puncture and then withdrawn.

FIG. 4C shows central portion 212 and deflectable portion 213 inserted through the puncture and positioned at or near the surface of the pericardium P. Visualization element 240 is used to guide central portion 212 and deflectable portion 213 along the posterior aspect of the sternum S to the surface of the pericardium P. Deflectable portion 213 may be manipulated using steering control 216. Access element 250 is retracted within distal end 233 of deflectable portion 213.

FIG. 4D shows access element 250 extended from distal end 233 of deflectable portion 213, and advanced through the pericardium P to create an access site. For an access element 250 having a sharp tip 255, visualization element 240 is used to visualize access element 250 as it is advanced through pericardium P. For an access element 250 used with RF energy, visualization element 240 is used to visualize access element 250 as RF energy is passed through access element 250 to penetrate pericardium P. Access element 250 may be rotated so that a desired surface is visible to visualization element 240. Saline, contrast, medications, and/or other fluids may be introduced through access element 250 into the pericardial space.

FIG. 4E shows guidewire 205 passed through access element 250 and positioned in the pericardial space.

FIG. 4F shows access element 250 retracted back into distal end 233 of deflectable portion 213, and central portion 212 and deflectable portion 213 withdrawn, leaving guidewire 205 in place.

FIG. 4G shows a sheath 280 advanced over guidewire 205 through the puncture and the access site and into the pericardial space. Other devices or guidewires may be advanced through sheath 280 to access the pericardial space. Saline, contrast, medications, and/or other fluids may be introduced through sheath 280 into the pericardial space.

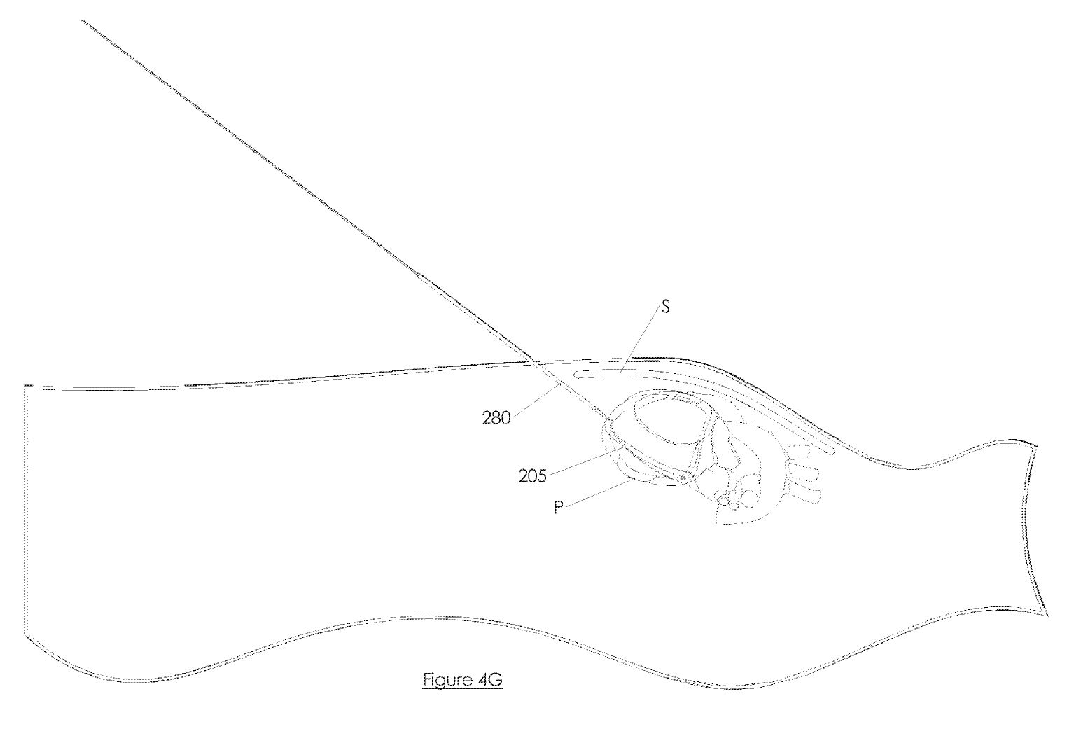

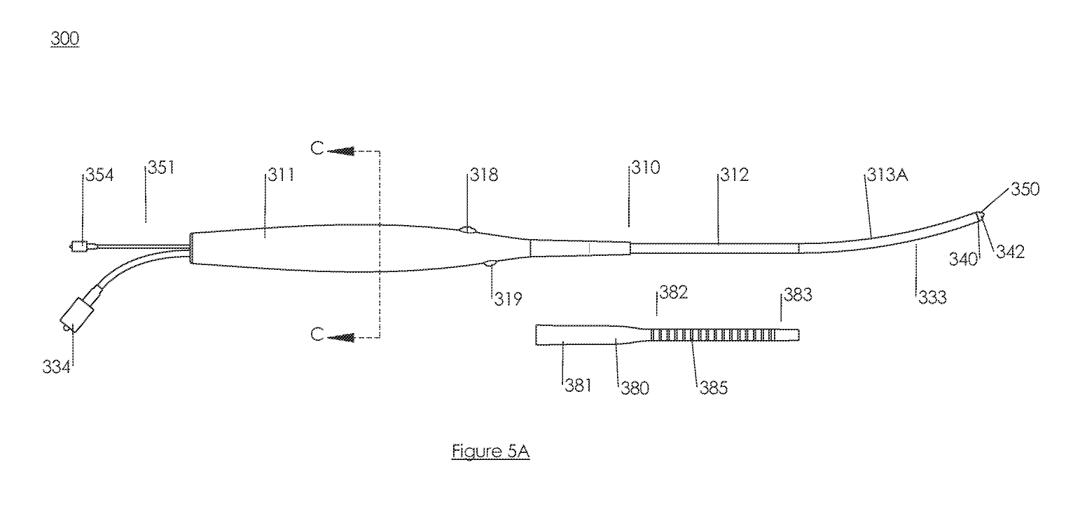

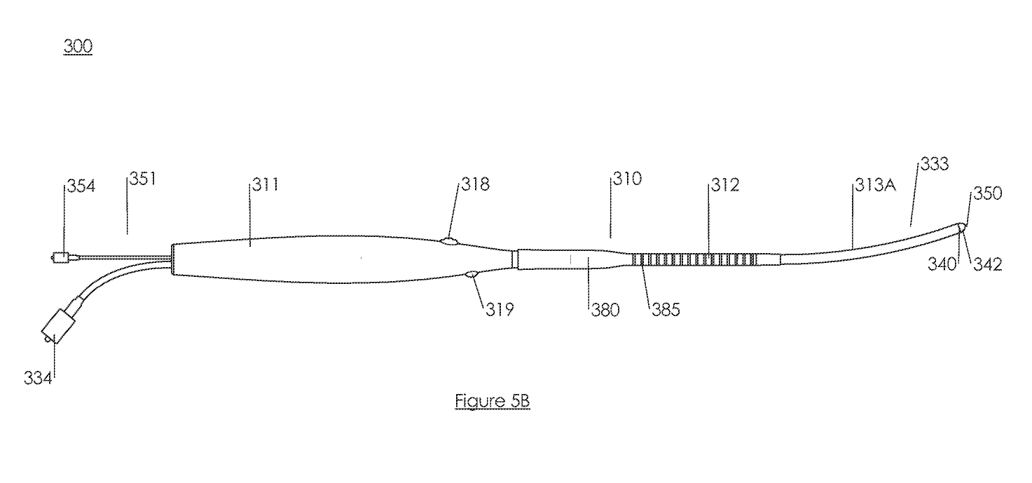

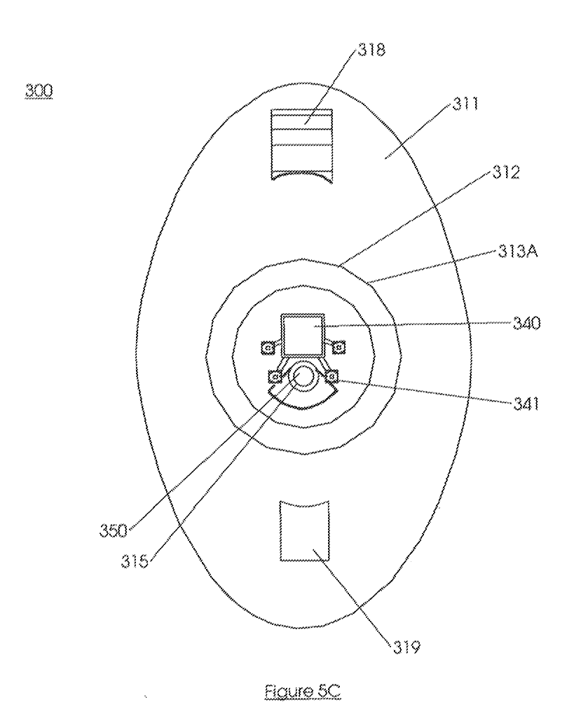

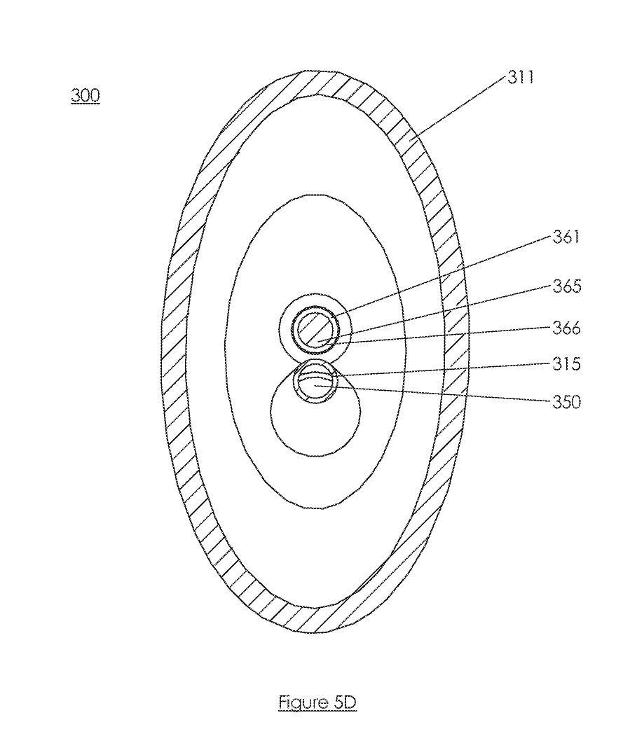

FIGS. 5A-5B show side views of yet another embodiment of an access device 300. FIG. 5C shows an end view of access device 300. FIG. 5D shows a cross-sectional end view of access device 300.

Access device 300 includes a housing 310, a visualization element 340, and an access element 350. Access device 300 may also include a sheath 380.

Housing 310 includes a handle 311, a central portion 312, and a distal portion 313A. Housing 310 also includes an access lumen 315 and a visualization lumen 361. Handle 311 includes a visualization control 318 and a light control 319. Handle 211 may also include a coupling 334 for attachment of a power source and a video monitor. Central portion 312 is coupled to handle 311, and is configured to be inserted into a puncture and navigate inside the body. Central portion 312 may be substantially rigid.

Distal portion 313A is coupled to central portion 312 and is also configured to be inserted into a puncture and navigate inside the body. Distal portion 313A may also be substantially rigid. Distal portion 313A may have a curved configuration, and may be bent or otherwise configured by the user and hold its shape. Distal portion 313A may include tubing made of a ductile material such as stainless steel or any other suitable material. Distal portion 313A includes a visualization element 340 and one or more lights 341.

Visualization element 340 and lights 341 may be coupled to a distal end 333 of distal portion 313A. Alternatively, visualization element 340 and lights 341 may be coupled to the side or any other suitable location of distal portion 313A. Visualization element 340 and lights 341 are coupled to visualization wires 365 and light wires 366 which pass through visualization lumen 361 to coupling 334. Visualization element 340, lights 341, and access lumen 315 are covered by a lens 342. Lens 342 includes an opening 343 which is continuous with access lumen 315. Lens 342 may also include a nozzle or other opening configured to clean lens 342. Lens 342 may have a hydrophobic coating or other coating to reduce adhesion of natural and synthetic materials that would obscure the image. Visualization element 340 may be turned on or off, or capture pictures or video using visualization control 318. Lights 341 may be turned on or off, or their intensity adjusted using light control 319.

Access element 350 is at least partially positioned within access lumen 315, and can slide and rotate within access lumen 315. Access element 350 may be used for injection of a liquid, passing of a guidewire 205, application of a vacuum, or any other suitable purpose. Access element 350 includes a proximal portion 351, a central portion 352, and a distal portion 353. Proximal portion 351 may include a coupling 354. Distal portion 353 has a tip 355 that may be a blunt tip trocar, a blunt tip obturator, a sharp edge trocar, a sharp edge needle (e.g., Tuohy, epidural, biopsy), a guidewire tip, or any other suitable instrument. Access element 350 may be configured to work with an RF, microwave, cryoablation, high intensity focused ultrasound (HIFU), laser, or any other suitable energy source. Distal portion 353 may have depth markings. Distal portion 353 may be connected to an ohmmeter to measure impedance as the needle penetrates the pericardial membrane into the pericardial space. The impedance measurement may be used to provide an indication as to whether the pericardial membrane has been penetrated. Access element 350 may be moved and rotated by manipulating proximal portion 351.

Central portion 352 is flexible, and capable of translating motions from proximal portion 351 to distal portion 353. Flexible central portion 352 allows access element 350 to move with distal portion 313A of housing 310. Central portion 352 may be constructed of a flexible braided material, a ductile metal, or any other suitable material. Proximal portion 351 may be substantially rigid. Distal portion 353 may be substantially rigid to facilitate penetration of tissue. Proximal portion 351, central portion 352, and distal portion 353 may be coupled with any suitable coupling device or method.

Sheath 380 includes a proximal portion 381, a central portion 382, and a distal portion 383. Proximal portion 381 may be grasped, and may include a coupling for attachment to an RF or other suitable energy source. Distal portion 383 may be made of a soft, flexible material and may stretch to fit snugly around housing 310. Central portion 382 may include electrodes 385 for coagulation and other purposes. Central portion 382 may have electrodes 385 that are configured circumferentially. Alternatively, electrodes 385 may be configured in a spiral, double helix, opposing helix, or any other suitable configuration. Electrodes 385 may be embedded in central portion 382 or otherwise coupled to central portion 382 in any suitable manner.

Sheath 380 may have a distal portion 383 that is tapered, with smaller end that tapers up in size towards central portion 382. The smaller end facilitates insertion of distal portion 383 into a puncture. The taper allows distal portion 383 to dilate the puncture as it is advanced. Electrodes 385 are configured to control bleeding proximate to the sheath at the site of the puncture, pericardium, or other structures.

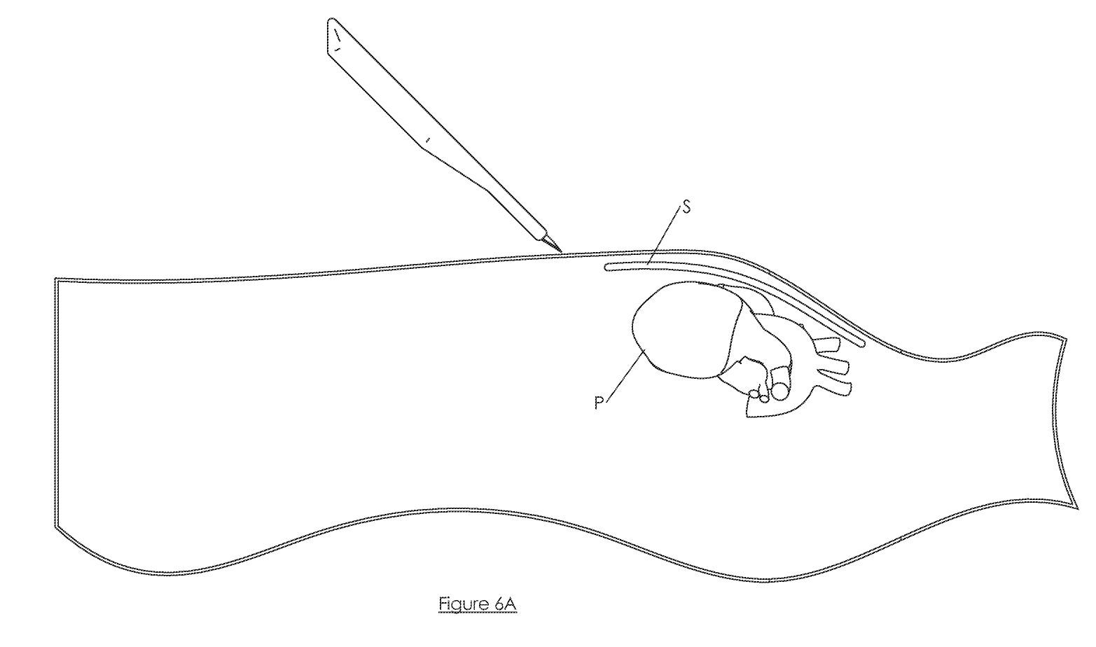

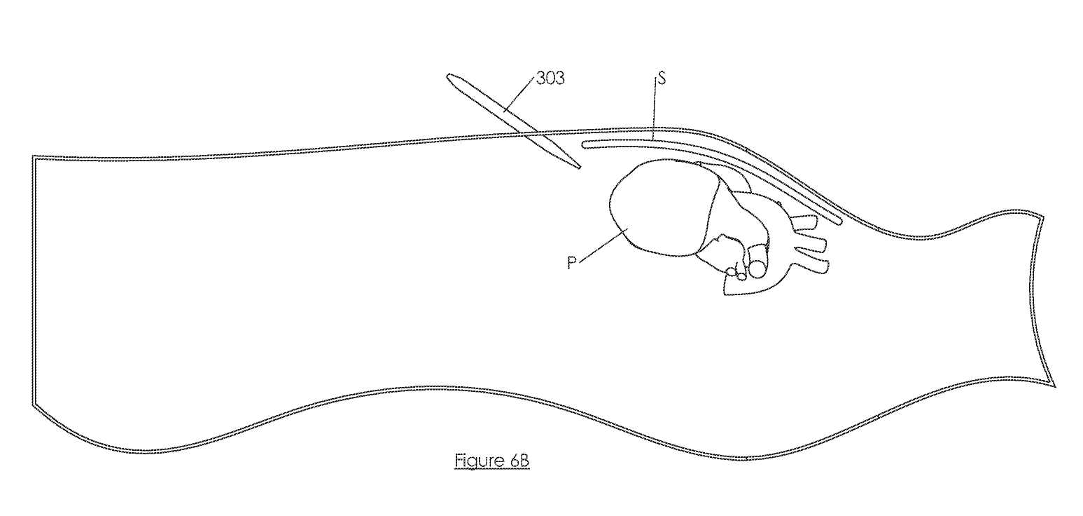

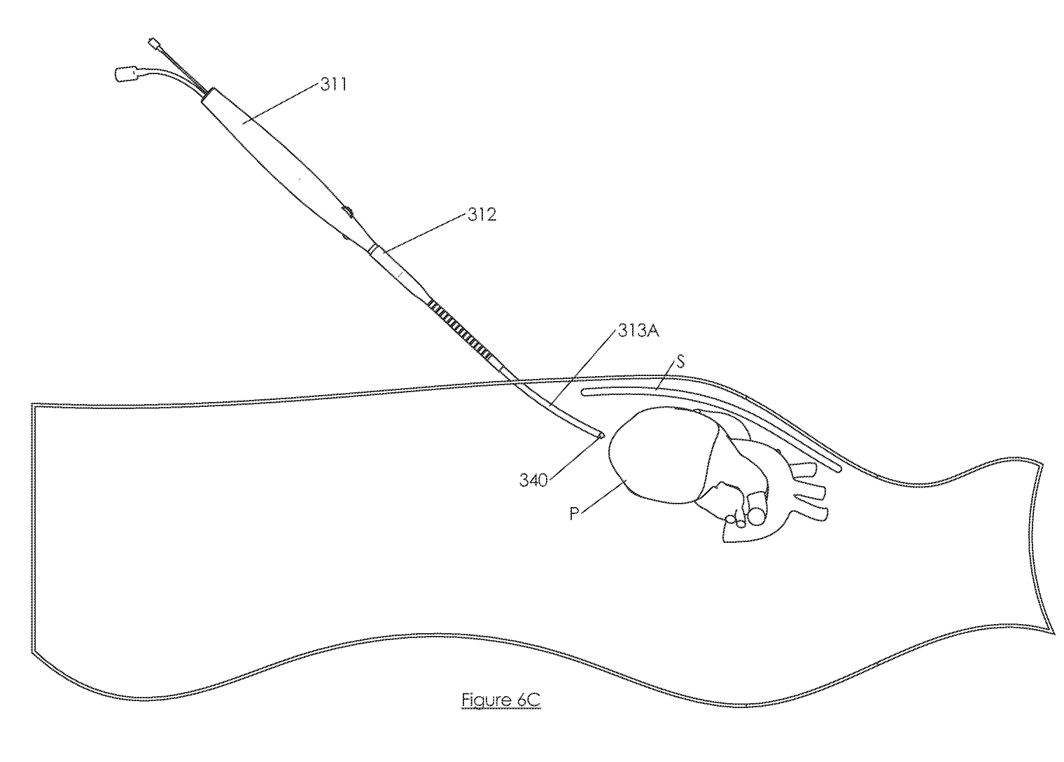

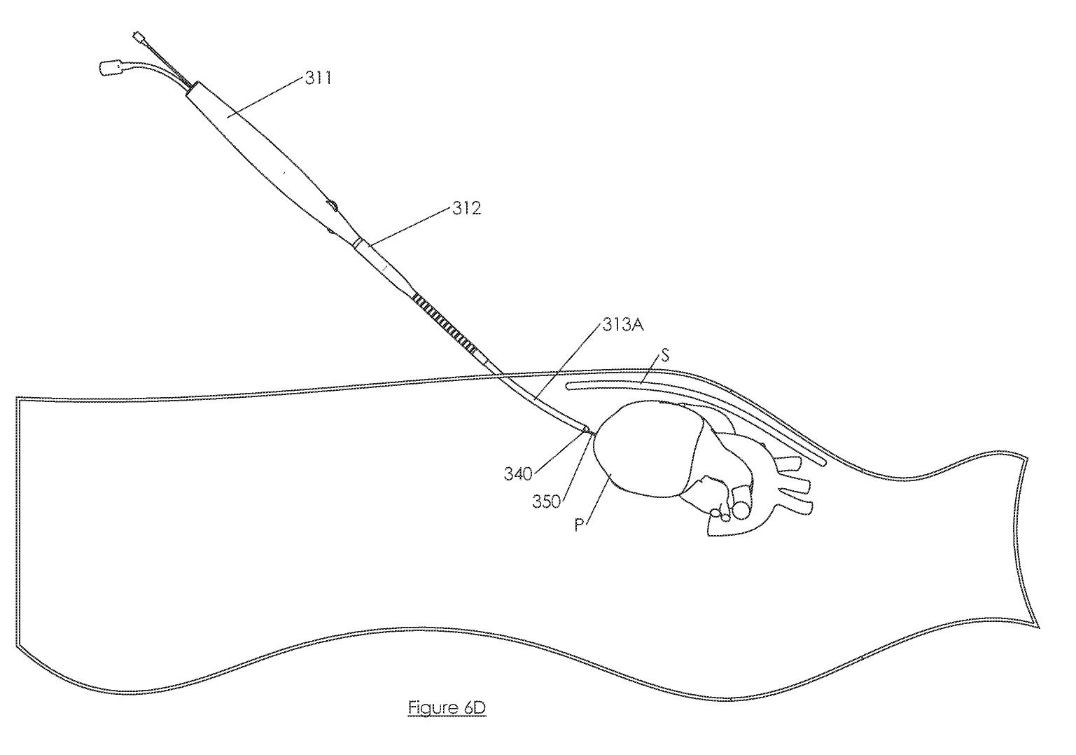

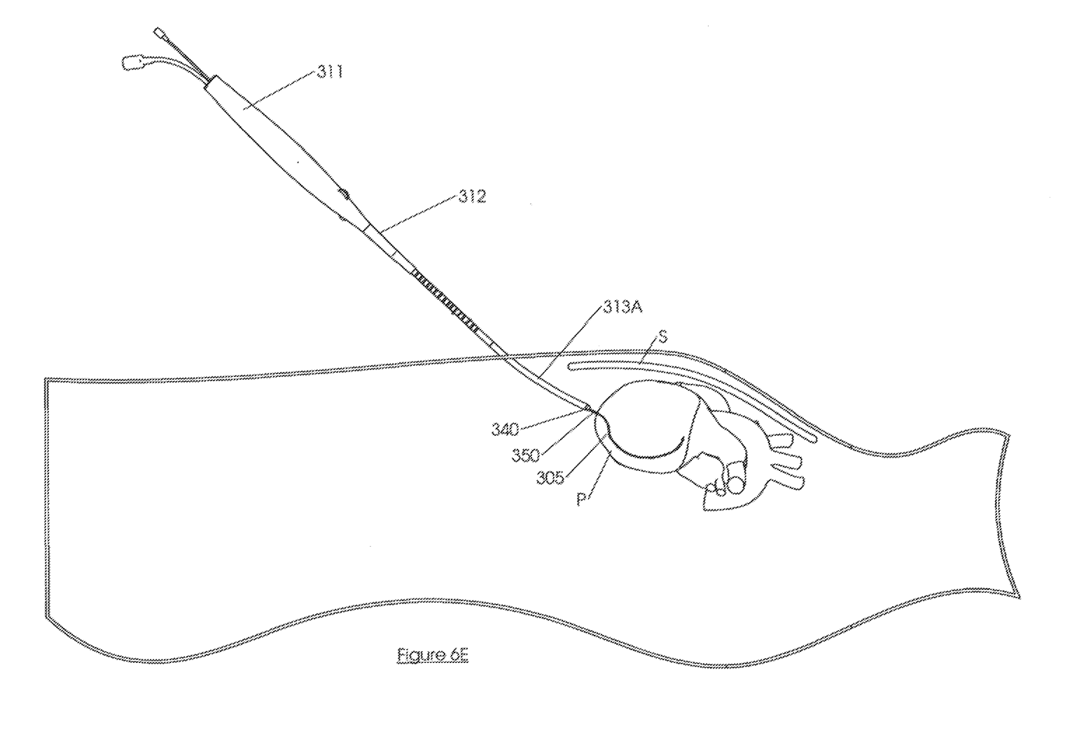

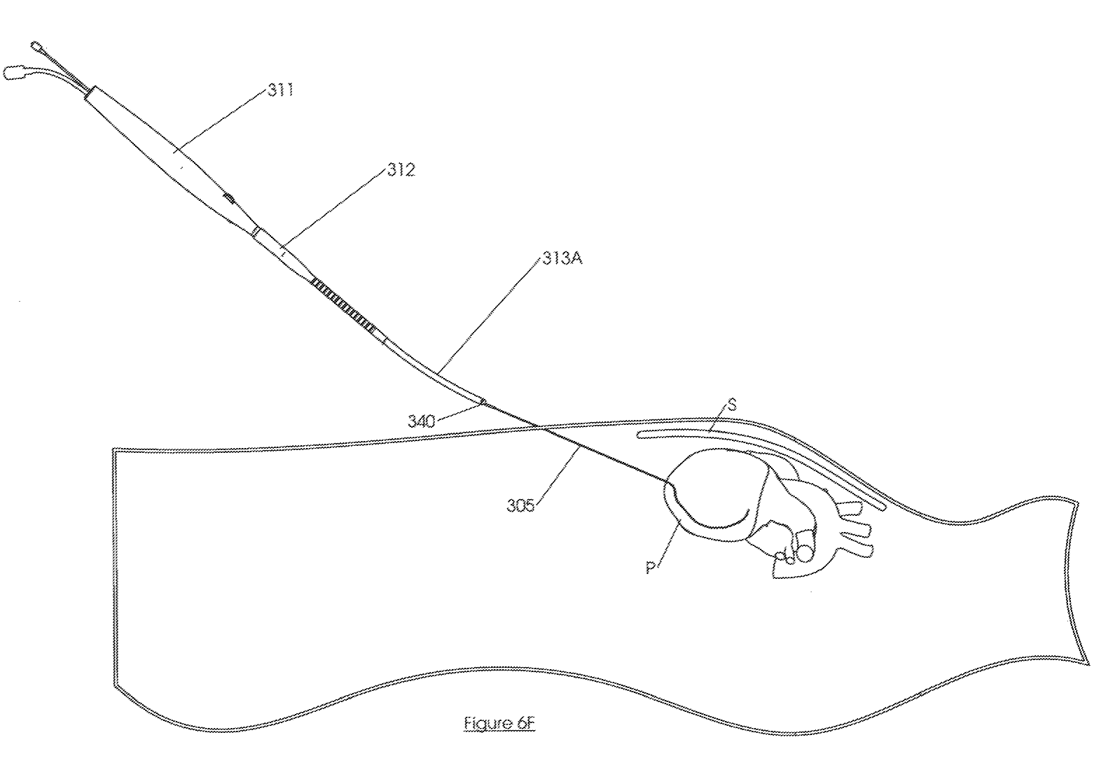

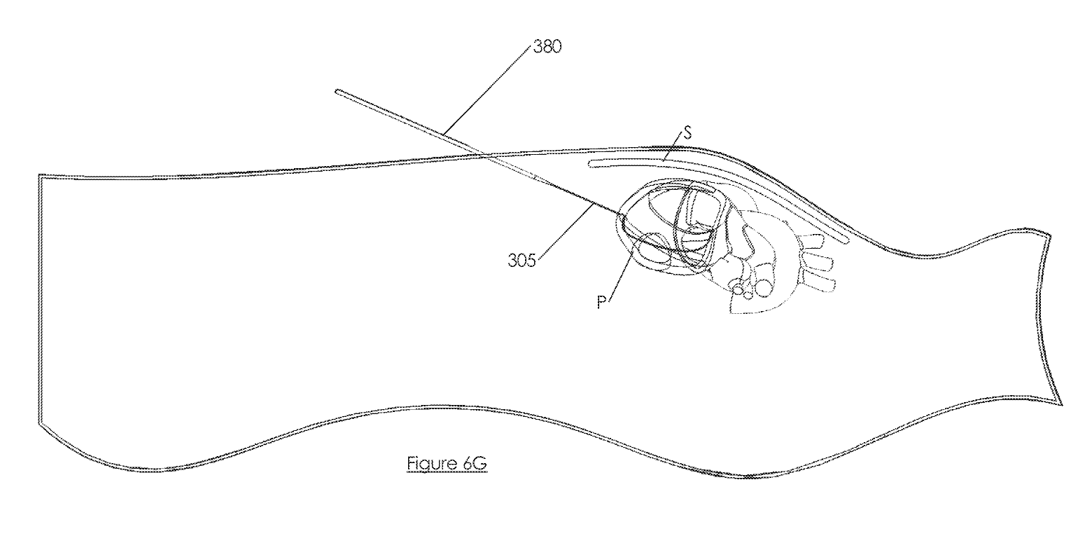

FIGS. 6A-6G show one method of using access device 300.

FIG. 6A shows a percutaneous puncture being made for a subxiphoid approach. Alternatively, an intercostal, apical, subclavian, suprasternal, or any other suitable approach may be used.

FIG. 6B shows a dilator 303 inserted through the puncture. Dilator 303 is used to dilate the puncture and then withdrawn.

FIG. 6C shows central portion 312 and distal portion 313A inserted through the puncture and positioned at or near the surface of the pericardium P. Visualization element 340 is used to guide central portion 312 and distal portion 313A along the posterior aspect of the sternum S to the surface of the pericardium P. Access element 350 is retracted within distal end 333 of distal portion 313A.

FIG. 6D shows access element 350 extended from distal end 333 of distal portion 313A, and advanced through the pericardium P to create an access site. For an access element 350 having a needle tip 355, visualization element 340 is used to visualize access element 350 as it is advanced through pericardium P. For an access element 350 used with RF energy, visualization element 340 is used to visualize access element 350 as RF energy is passed through access element 350 to penetrate pericardium P. Access element 350 may be rotated so that a desired surface is visible to visualization element 340. Saline, contrast, medications, and/or other fluids may be introduced through access element 350 into the pericardial space.

FIG. 6E shows guidewire 305 advanced through access element 350 and positioned in the pericardial space.

FIG. 6F shows access element 350 retracted back into distal end 333 of distal portion 313A, and central portion 312 and distal portion 313A withdrawn, leaving guidewire 305 in place.

FIG. 6G shows sheath 380 advanced over guidewire 305 through the access site and into the pericardial space. Electrodes 385 may be used for coagulation. Other devices or guidewires may be advanced through sheath 380 to access the pericardial space. Saline, contrast, medications, and/or other fluids may be introduced through sheath 380 into the pericardial space.

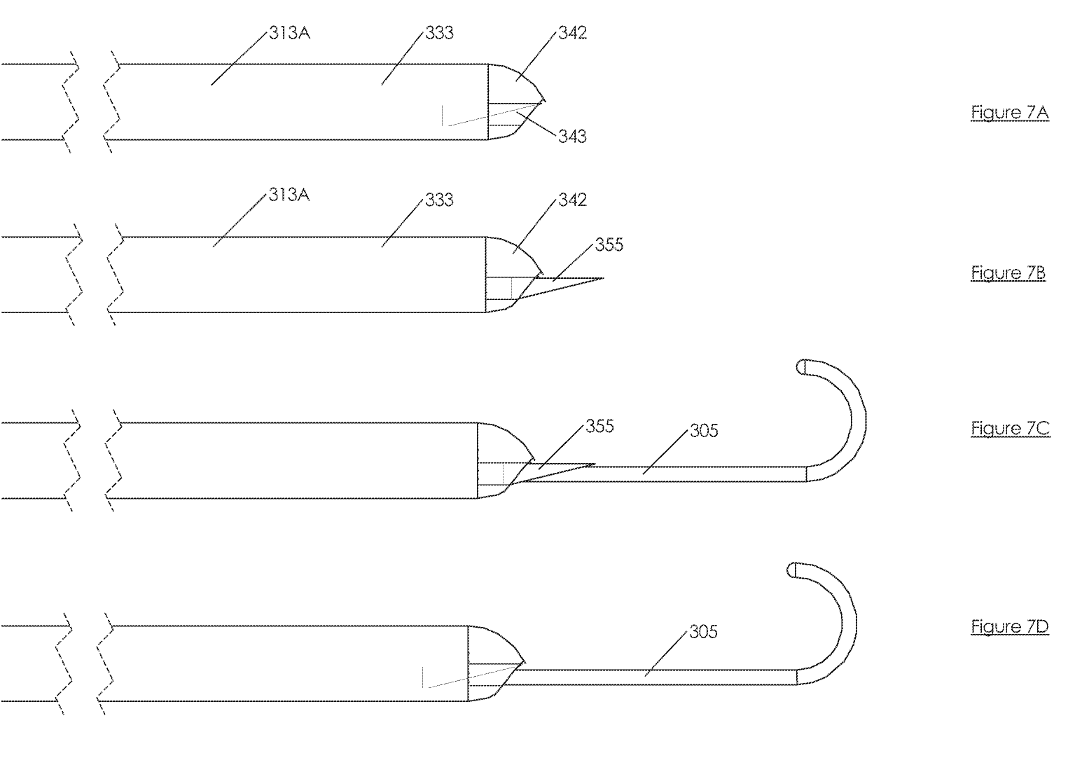

FIGS. 7A-7D show enlarged cross-sectional side views of distal portion 313A. FIG. 7A shows distal portion 313A with tip 355 of access element 350 retracted inside distal end 333. FIG. 7B shows distal portion 313A with tip 355 of access element 350 extended from distal end 333. FIG. 7C shows guidewire 305 advanced through access element 350. FIG. 7D shows tip of 355 of access element 350 pulled back inside distal end 333. Guidewire 305 remains in place.

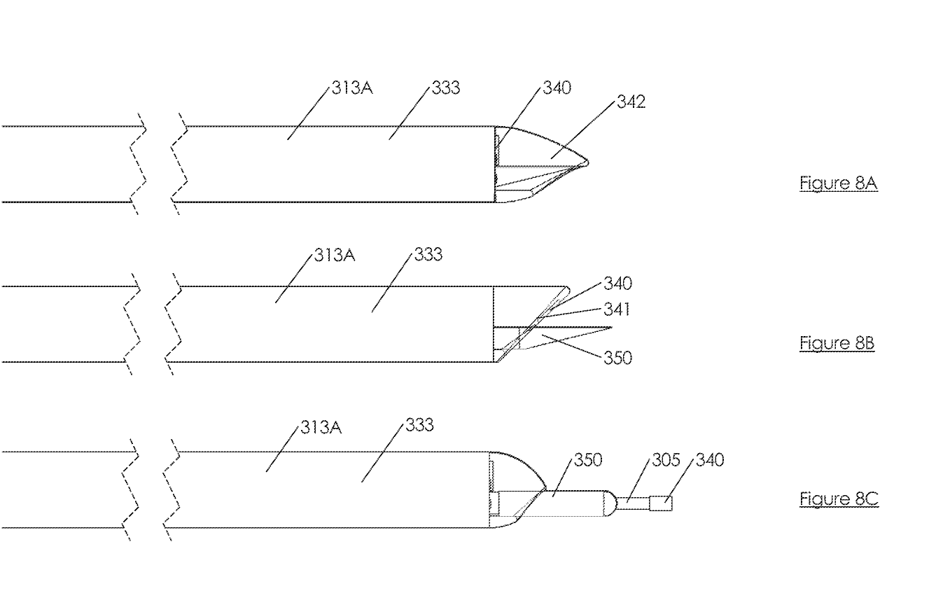

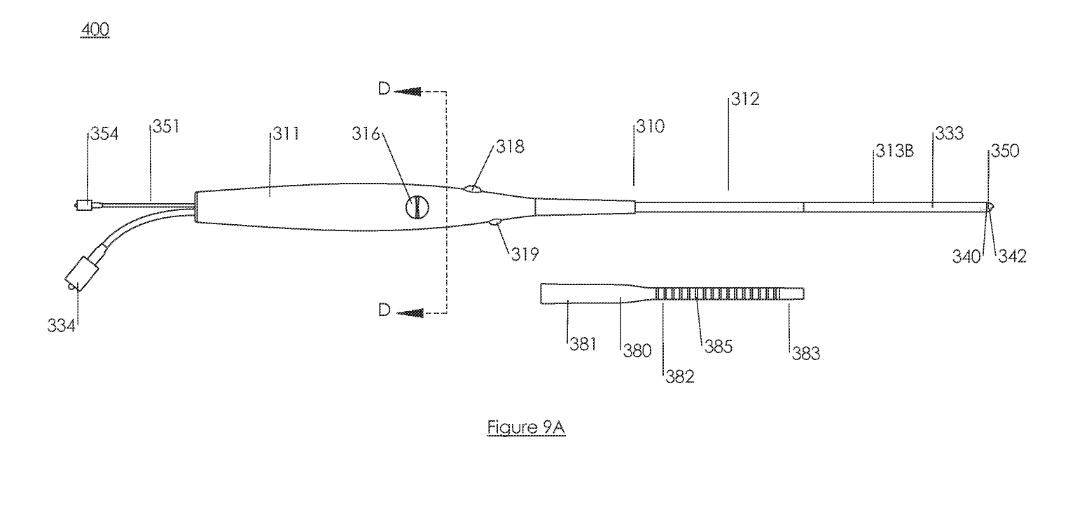

FIGS. 8A-8C show other embodiments of distal end 333 of distal portion 313A. FIG. 8A shows another embodiment of distal end 333 with lens 342 having a tapered profile. The tapered profile of distal end 333 may facilitate its advancement into the pericardial space. FIG. 8B shows yet another embodiment of distal end 333 having an asymmetrical tapered profile. Visualization element 340 and lights 341 may be mounted on the underside of the taper facing access element 350. The tapered profile of distal end 333 may facilitate its advancement into the pericardial space. FIG. 8C shows still another embodiment of distal end 333 with a visualization element 340 mounted on guidewire 305 and positioned within access element 350. Visualization element 340 is capable of being moved independently of access element 350.

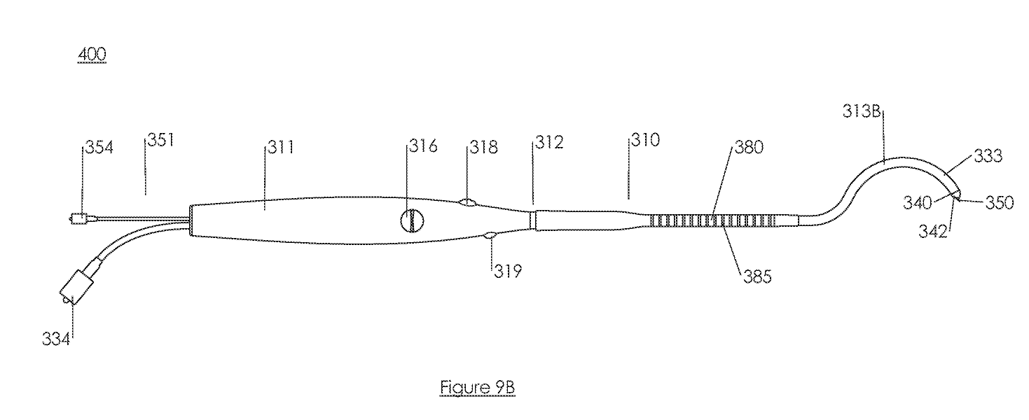

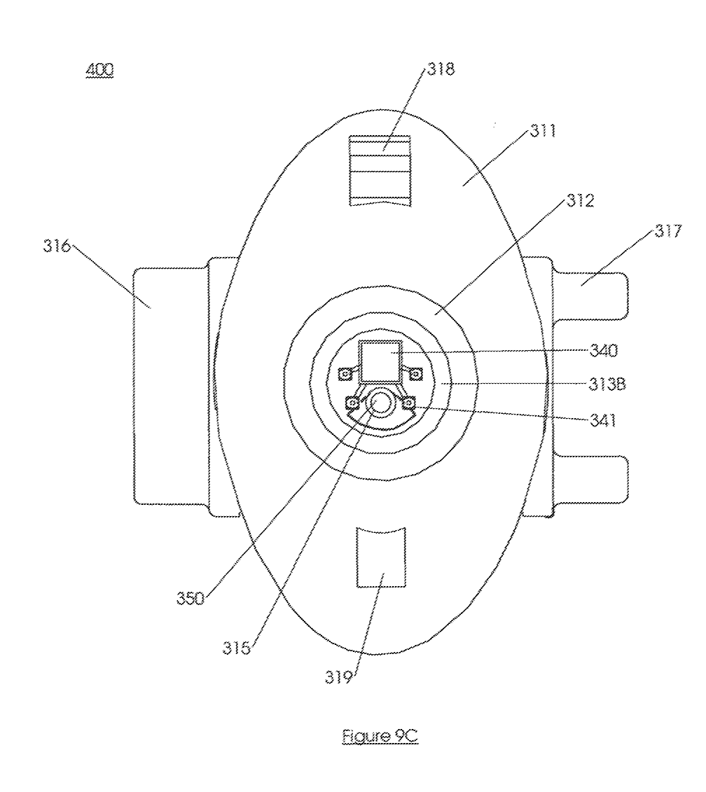

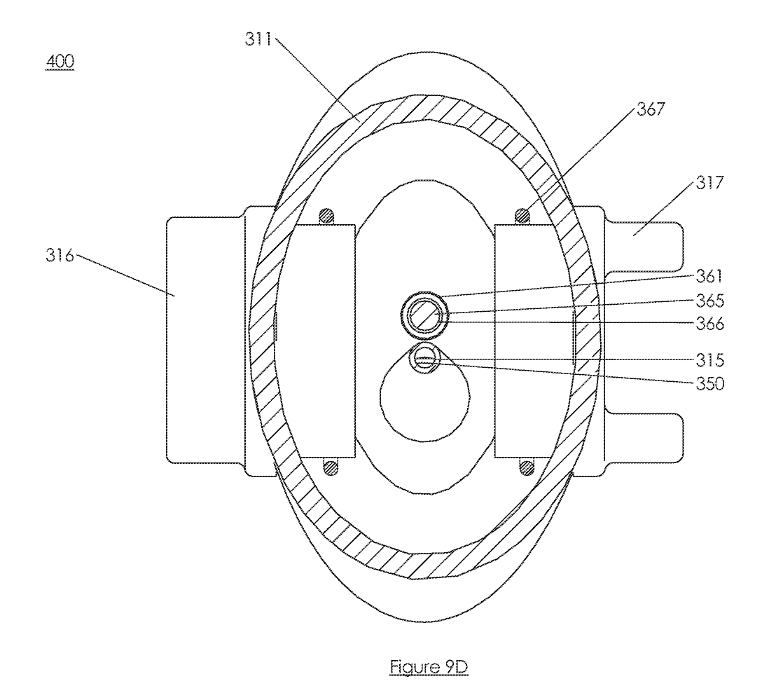

FIGS. 9A-9B show side views of still another embodiment of access device 400. FIG. 9C shows an end view of access device 400. FIG. 9D shows a cross-sectional end view of access device 400.

Access device 400 includes a housing 310, a visualization element 340, and an access element 350. Access device 400 may also include a sheath 380.

Housing 310 includes a handle 311, a central portion 312, and a deflectable portion 313B. Housing 310 also includes an access lumen 315 and a visualization lumen 361. Handle 311 includes a steering control 316, a tension lock 317, a visualization control 318, and a light control 319. Handle 211 may also include a coupling 334 for attachment of a power source and a video monitor. Central portion 312 is coupled to handle 311, and is configured to be inserted into a puncture and navigate inside the body. Central portion 312 may be soft and flexible, or more rigid depending on the application and user preferences.

Deflectable portion 313B is coupled to central portion 312 and is also configured to be inserted into a puncture and navigate inside the body. Deflectable portion 313B may be deflected in one or more axes, as shown for example in FIG. 5B. Deflectable portion 313B may be controlled with pullwires 367 coupled to steering control 316. Deflectable portion 313B may be locked in a desired configuration using tension lock 317. Deflectable portion 313B includes a visualization element 340 and one or more lights 341.

Access device 400 is similar to access device 300, but instead of a distal portion 313A that may be bent or otherwise configured by the user before being introduced into the body, access device 400 includes a deflectable portion 313B that is controlled by pullwires 367 coupled to steering control 316 and tension lock 317. The remainder of access device 400 is similar to access device 300. Access device 400 may be used in a manner similar to access device 300.

Access device 400 may have a central portion 312 that is lengthened. Access device 400 with a lengthened central portion 312 may be used to visualize and treat structures in the mediastinal space outside of the pericardium. Access device 400 with a lengthened central portion 312 may used to first create an entry site through the pericardium and introduce guidewire 305 into the pericardial space. Deflectable portion 313B may then be advanced over guidewire 305 through the entry site and into the pericardial space. Deflectable portion 313B may then be steered and navigated within the pericardial space to find a desired exit site. Deflectable portion 313B may then be used to create an exit site through the pericardium and access structures in the mediastinal space outside of the pericardium. Structures located posterior of the heart, superior to the diaphragm, and inferior to the clavicle such as the esophagus, trachea, primary bronchi, posterior pleural cavities, thoracic vertebrae and other structures may thus be accessed for delivery of therapeutics, biopsy, fixation, ablation, survey, and other purposes.

Visualization element 140, 240, 340 may be a CCD, CMOS, or any other suitable imaging device, such as those available Omnivision Technologies, Inc., Santa Clara, Calif. Alternatively, visualization element 140, 240, 340 may be a fiber optic device. Visualization element 140, 240, 340 may also be an IntroSpicio 120 CMOS camera, available from Medigus Ltd., Omer, Israel.

Although the above embodiments and methods describe using the access device to visualize and access the pericardial space, this device may be used to visualize and access any space, tissue, or organ in the body. Examples include the heart, peritoneum, diaphragm, mediastinal structures, and abdominal organs.

While the foregoing has been with reference to particular embodiments of the invention, it will be appreciated by those skilled in the art that changes in these embodiments may be made without departing from the principles and spirit of the invention.

All publications and patent applications mentioned in this specification are herein incorporated by reference to the same extent as if each individual publication or patent application was specifically and individually indicated to be incorporated by reference.

1. A device for accessing a pericardial space, the device comprising:
a housing having a handle, a central portion coupled to the handle, a distal portion coupled to the central portion, and a longitudinal access lumen, the central portion and the distal portion being configured to be inserted through a percutaneous puncture and navigate within a body;
an access element at least partially positioned within the access lumen, the access element capable of being extended and retracted from a distal end of the distal portion of the housing, the access element capable of penetrating a pericardium surrounding the pericardial space to create an access site for accessing the pericardial space; and
a visualization element coupled to the distal portion of the housing, the visualization element capable of aiding navigation within the body and visualizing the access element.

2. The device of claim 1, wherein the distal portion is substantially rigid.

3. The device of claim 2, wherein the distal portion is curved.

4. The device of claim 1, wherein the distal portion is made of a ductile material.

5. The device of claim 1, wherein the distal portion is deflectable with a steering control coupled to the distal portion.

6. The device of claim 1, wherein the access element comprises:
a proximal portion that is substantially rigid;
a central portion coupled to the proximal portion, the central portion being substantially flexible and capable of being deflected with the deflectable portion; and
a distal portion coupled to the central portion, the distal portion being substantially rigid.

7. The device of claim 1, further comprising:
an RF energy source coupled to the access element, the access element capable of transmitting an RF energy to assist in penetrating the pericardium.

8. The device of claim 1, further comprising:
a lens coupled to the distal portion of the housing, the lens covering the visualization element.

9. The device of claim 1, further comprising:
a sheath slidably coupled to the central portion of the housing, the sheath including a coagulation electrode for controlling bleeding.

10. A device for accessing a space within a body, the device comprising:
a housing having a handle, a central portion coupled to the handle, a distal portion coupled to the central portion, and a longitudinal access lumen, the central portion and the distal portion being configured to be inserted through a percutaneous puncture and navigate within the body;
an access element at least partially positioned within the access lumen, the access element capable of being extended and refracted from a distal end of the distal portion of the housing, the access element capable of penetrating a tissue surrounding the space to create an access site for accessing the space; and
a visualization element coupled to the distal portion of the housing, the visualization element capable of aiding navigation within the body and visualizing the access element.

11. The device of claim 10, wherein the distal portion is substantially rigid.

12. The device of claim 11, wherein the distal portion is curved.

13. The device of claim 10, wherein the distal portion is made of a ductile material.

14. The device of claim 10, wherein the distal portion is deflectable with a steering control coupled to the distal portion.

15. The device of claim 10, wherein the access element comprises:
a proximal portion that is substantially rigid;
a central portion coupled to the proximal portion, the central portion being substantially flexible and capable of being deflected with the deflectable portion; and
a distal portion coupled to the central portion, the distal portion being substantially rigid.

16. The device of claim 10, further comprising:
an RF energy source coupled to the access element, the access element capable of transmitting an RF energy to assist in penetrating the tissue.

17. The device of claim 10, further comprising:
a lens coupled to the distal portion of the housing, the lens covering the visualization element.

18. The device of claim 10, further comprising:
a sheath slidably coupled to the central portion of the housing, the sheath including a coagulation electrode for controlling bleeding.

19. (canceled)

20. A method for accessing a space within a body, the method comprising:
providing a housing having a handle, a central portion coupled to the handle, a distal portion coupled to the central portion, and a longitudinal access lumen;
providing an access element at least partially positioned within the access lumen, the access element being retracted within the access lumen;
providing a visualization element coupled to a distal portion of the housing;
inserting the central portion and the distal portion through a percutaneous puncture;
navigating within the body with the visualization element;
locating a tissue surrounding the space with the visualization element;
extending the access element from the distal portion of the housing; and
penetrating the tissue with the access element to create an access site, while visualizing the access element and the tissue with the visualization element.

21. The method of claim 20, wherein penetrating the tissue includes passing an RF energy through the access element.

22-32. (canceled)
